(19) United States
(12) Patent Application Publication
(10) Pub. No.: US 2023/0087685 A1
(43) Pub. Date: Mar. 23, 2023

(54) APPARATUS AND METHOD FOR SPECTRAL DOMAIN OPTICAL IMAGING

(71) Applicant: CYLITE PTY LTD, Notting Hill, Victoria (AU)

(72) Inventor: Steven James FRISKEN, Vaucluse (AU)

(21) Appl. No.: 17/795,842

(22) PCT Filed: Jan. 31, 2021
(86) PCT No.: PCT/AU2021/050067

§ 371 Date: Jul. 27, 2022

(30) Foreign Application Priority Data
Jan. 31, 2020 (AU) 2020900264

(51) Int. Cl. A61B 3/10; G01J 1/60; G01J 3/28; G01J 3/02; G01B 9/02091; A61B 3/00; A61B 3/12; A61B 3/14

(52) U.S. Cl. CPC A61B 3/102; G01J 1/60; G01J 3/2803; G01J 3/0229; G01J 3/0208; G01B 9/02091; A61B 3/0008; A61B 3/1241; A61B 3/14; G01J 2003/2813

(57) ABSTRACT

Apparatus and methods are presented for spectral domain optical imaging, in particular for single shot 3-D spectral domain imaging of the retina of the human eye. In certain embodiments one or more 3-D images across elongated areas of an object are acquired, with scanning perpendicular to the long axis of the elongated areas for imaging extended volumes of the object. In preferred embodiments the captured light is sampled in the Fourier plane, in a dimension substantially perpendicular to the long axis, with a cylindrical lenslet array, while in other embodiments the captured light is sampled in the image plane. Apparatus and methods are also presented for hyperspectral imaging of the retina, with the illuminating beams preferably angled to suppress interference from corneal reflections. Apparatus and methods are also presented for multi-wavelength wavefront sensing, with simultaneous capture of light in two or more paths with different delays.

### FIELD OF THE INVENTION

The invention relates to apparatus and methods for spectral domain optical imaging, in particular for 3-D spectral domain imaging of the retina of the human eye. However it will be appreciated that the invention is not limited to this particular field of use.

### RELATED APPLICATIONS

The present application claims priority from Australian Provisional Patent Application No 2020900264 filed on 31 Jan. 2020, the contents of which are incorporated herein by reference.

### BACKGROUND OF THE INVENTION

Any discussion of the prior art throughout the specification should in no way be considered as an admission that such prior art is widely known or forms part of the common general knowledge in the field.

Optical coherence tomography (OCT) is a widely used interferometric technique for studying biological samples including in-vivo tissue such as the human eye, with lateral and depth resolution, using information contained in the amplitude and phase of light reflected or scattered from the sample. Most current OCT systems utilise spectral domain techniques where the depth information is encoded in the spectral response of the interference signal, which can be recorded as a time-varying function of wavelength (swept source OCT) or by dispersing the interference signal and detecting the different wavelengths simultaneously along a detector array (spectrometer-based OCT).

Many OCT systems acquire depth-resolved data at discrete points, i.e. A-scans, and use beam scanning in one or two lateral dimensions to generate B-scans (depth-resolved data in one lateral dimension) or C-scans (depth-resolved data in two lateral dimensions). Faster acquisition is generally desirable irrespective of the type of scan, especially for reducing motion-induced artefacts with in-vivo samples. So-called ‘snap shot’ spectrometer-based OCT approaches, in which depth-resolved data from a slice or volume of an object is acquired in a single frame of a sensor array, are particularly advantageous for reducing motion-induced artefacts. This is in contrast to swept source OCT approaches relying on sweeping a wavelength and capturing multiple frames corresponding to each of the wavelengths, which are highly sensitive to submicron shifts of the object during acquisition. Published US patent application No 2014/0028974 A1 entitled ‘Line-field holoscopy’ discloses a single-shot B-scan technique in which cylindrical lenses produce a line illumination on an object and on a reference mirror, followed by dispersion of the combined return sample and reference beams along one axis of a two-dimensional (2-D) sensor array. Advantageously, the holoscopic nature of this technique, where the sensor array is imaged to the far field of the object, allows digital refocusing and aberration correction as well as a degree of confocal rejection. However for full 3-D (C-scan) imaging the illuminated line needs to be scanned in the orthogonal direction and the 2-D sensor array read out repeatedly, and with in-vivo samples it is generally difficult to maintain phase stability between the consecutive B-scans. Consequently the achievable resolution and depth of focus tends to be highly anisotropic.

As disclosed in published US patent application No 2016/0345820 A1 entitled ‘High resolution 3-D spectral domain optical imaging apparatus and method’, the contents of which are incorporated herein by reference, single-shot C-scan acquisition can be achieved if a 2-D area of an object is illuminated and the combined returning sample and reference wavefronts sampled in two lateral dimensions, e.g. with a 2-D lenslet array, and the resulting sampling points dispersed onto separate sets of pixels of a 2-D sensor array. Digital refocusing can then be applied to the tomograms. The lateral resolution depends largely on the NA of the objective lens and may for example be around 3 μm. A limitation with this technique is that the illuminated area on the object needs to be relatively small, of order 100 μm×100 μm, to reduce the impact of multiple scattering and because of the limited number of sampling points offered by commercially available 2-D lenslet arrays. Tomograms of multiple adjacent or overlapping volumes can be acquired by moving the object relative to the illuminated area in one or two dimensions, then stitched together to image larger object volumes, with the overall acquisition speed limited by the frame rate of the 2-D sensor array.

Other disadvantages of the prior art include the presence of cross-talk signals creating artefacts or degradation of signal-to-noise ratio (SNR) induced by multiple scattering of light that is in part coherent with the signal being measured. Furthermore the acceptance NA of existing systems is relatively low, meaning only a small fraction of the scattered light is contributing to the signal so the power densities required for a given SNR need to be increased to compensate.

Hyperspectral imaging provides various contrast mechanisms for screening and assessment of a range of retinal pathologies by measuring reflected/scattered light or fluorescence spectra over a range of discrete wavelengths, typically 20 to 100 wavelengths over the range of the visible or near-IR. However existing hyperspectral imaging systems based on fundus cameras and illumination with various wavelengths have limitations in the interpretation of acquired spectral data because of the inability to discriminate the signal from background light that may arise from reflection from other surfaces, in particular from the cornea. Additionally, sequential acquisition of wavelengths provides challenges for alignment of the different frames, which can cause errors in the predicted spectra from each pixel. Furthermore conventional instruments for acquiring hyperspectral images of the retina require the pupil to be dilated, which increases the time required and is inconvenient for the patient.

Many of the important applications of OCT technology have been related to ocular imaging and in particular to imaging of the fundus over a wide field, which has proven to be a particular challenge due to the limited acquisition speed of scanning systems and their ability to register scans. This is especially apparent in OCT-A (Angiography) applications where the time variation in an OCT signal is used as an indicator of blood flow. OCT-A systems need to perform repeated measurements over a typical time interval of a few milliseconds at each required location on the retina. This generally implies slow and uncomfortable acquisitions when covering a very wide field of the retina, say >75 degrees, often with compromised spatial resolution, as well as requiring extremely demanding registration of the scans to ensure that bulk motion of the eye is not misinterpreted as flow. Wide-field OCT-A images are hence prone to motion artifacts, often in the form of registration stripes.

OCT systems commonly utilise broadband sources such as superluminscent LEDs or rapidly swept lasers, i.e. sources with relatively low spectral coherence, for providing depth resolution in the interference signal. Although spatially incoherent sources can be used in full-field time domain OCT, time domain systems typically have reduced SNR compared to spectral domain OCT systems as well as additional noise when away from the focal plane, limiting their performance. Furthermore, typical commercially available OCT systems have been based on point scanning, utilising sources with high spatial coherence for capture of phase information. This poses quite low limits on the illumination levels which can be used in ocular imaging, because the light from such sources can be focused to small and therefore high intensity spots on the retina, consequently limiting the area that can be imaged with a good SNR.

Unless the context clearly requires otherwise, throughout the description and the claims the words ‘comprising’, ‘comprises’ and the like are to be construed in an inclusive sense as opposed to an exclusive or exhaustive sense. That is, they are to be construed in the sense of ‘including, but not limited to’. Similarly, unless the context clearly requires otherwise, the word ‘or’ is to be construed in an inclusive sense rather than an exhaustive sense. That is, the expression ‘A or B’ is to be construed as meaning ‘A, or B, or both A and B’.

### OBJECT OF THE INVENTION

It is an object of the present invention to overcome or ameliorate at least one of the limitations of the prior art, or to provide a useful alternative. It is an object of the present invention in a preferred form to provide spectral domain OCT apparatus and methods for single shot acquisition of 3-D images of laterally elongated volumes of an object, and for imaging extended volumes of the object by scanning the illumination in one lateral dimension. It is another object of the present invention in a preferred form to provide apparatus and methods for in-vivo hyperspectral imaging of the retina with reduced interference from corneal reflections.

### SUMMARY OF THE INVENTION

According to a first aspect of the present invention there is provided an apparatus for imaging an eye, said apparatus comprising:
an illumination system comprising an optical source for illuminating, with a multi-wavelength optical beam, a volume of the eye, said volume to be imaged in three spatial dimensions, said volume having an elongated lateral cross-sectional area with a short axis, a long axis and an aspect ratio defined by the ratio of the long axis to the short axis;
an optical system for capturing and anisotropically transforming light scattered or reflected from the illuminated volume;
one or more beam splitters for splitting light emitted from said optical source into a reference beam and said multi-wavelength optical beam, and for combining said reference beam with the captured light;
a spatial sampling element for sampling the anisotropically transformed captured light in one dimension; and
a measurement system comprising a two-dimensional sensor array for simultaneous capture of phase and amplitude information over a range of wavelengths of the captured light sampled by said spatial sampling element.

Preferably, the optical system is configured to cooperate with the optical power elements of the eye, for imaging the retina of the eye. In certain embodiments the measurement system is configured to capture phase and amplitude information in first and second frames of said two-dimensional sensor array, for measurement of changes due to blood flow in the illuminated volume of the retina.

According to a second aspect of the present invention there is provided an optical imaging apparatus comprising:
an illumination system comprising an optical source for illuminating, with a multi-wavelength optical beam, a volume of an object, said volume to be imaged in three spatial dimensions, said volume having an elongated lateral cross-sectional area with a short axis, a long axis and an aspect ratio defined by the ratio of the long axis to the short axis;
an optical system for capturing and anisotropically transforming light scattered or reflected from the illuminated volume;
one or more beam splitters for splitting light emitted from said optical source into a reference beam and said multi-wavelength optical beam, and for combining said reference beam with the captured light;
a spatial sampling element for sampling the anisotropically transformed captured light in one dimension; and
a measurement system comprising a two-dimensional sensor array for simultaneous capture of phase and amplitude information over a range of wavelengths of the captured light sampled by said spatial sampling element.

The first and second aspects share a number of common preferments.

The optical system is preferably configured such that, in use, the aspect ratio of the anisotropically transformed light at the spatial sampling element is less than the aspect ratio of the lateral cross-sectional area of the illuminated volume.

Preferably, the spatial sampling element is positioned for Fourier plane sampling of the captured light. In preferred embodiments the spatial sampling element comprises a cylindrical lenslet array or a linear aperture array. The optical system and the spatial sampling element are preferably configured such that, in use, the captured light sampled by the spatial sampling element is projected onto a substantial portion of the two-dimensional sensor array, the substantial portion comprising at least 100 pixels in each dimension, more preferably at least 2000 pixels in each dimension.

Preferably, the optical source is at least partially spatially incoherent. In preferred embodiments the apparatus comprises an aperture for adjusting the spatial coherence of the light emitted from the optical source. The optical source or the aperture are preferably selected such that the multi-wavelength optical beam is substantially spatially coherent across the short axis of the lateral cross-sectional area. In certain embodiments the apparatus comprises an etalon for extending the coherence length of light emitted from the optical source.

The measurement system preferably comprises a dispersive element for dispersing the captured light in the direction substantially parallel to the sampling dimension of the spatial sampling element.

In certain embodiments the optical system comprises a series of cylindrical lenses forming a 4F relay system in the direction of the long axis and a 2F relay system in the direction of the short axis.

In certain embodiments the illumination system is configured such that, in use, the aspect ratio of the elongated lateral cross-sectional area is at least ten, or at least twenty, or at least fifty, or at least one hundred.

The apparatus may comprise one or more optical power elements for re-sizing the reference beam so as to increase the overlap between the reference beam and the captured light.

Preferably, the apparatus comprises a computer for processing the phase and amplitude information to construct a three-dimensional image of an optical characteristic of the eye or of the object over the illuminated volume. In certain embodiments the optical characteristic is selected from the group comprising phase, reflectivity, refractive index, refractive index changes and attenuation.

In certain embodiments the measurement system comprises a polarisation separation element for capturing phase and amplitude information for at least first and second polarisation states of the captured light. The optical characteristic may comprise birefringence or degree of polarisation. Preferably, the computer is configured to apply a focusing or aberration correction function to the phase and amplitude information.

In preferred embodiments the apparatus is configured to move the illuminated volume in a direction substantially perpendicular to the long axis of its elongated lateral cross-sectional area, for imaging larger volumes of the eye or of the object.

According to a third aspect of the present invention there is provided an apparatus for wavefront sensing comprising:
an illumination system comprising an optical source for illuminating, with a multi-wavelength optical beam, an area of an object;
an optical system for capturing an optical wavefront comprising light scattered or reflected from the illuminated area;
an element for splitting the captured light into two or more paths having different optical delays;
a spatial sampling element for simultaneously sampling, in one dimension, the captured light in the two or more paths; and
a measurement system comprising a two-dimensional sensor array for simultaneous measurement, over a range of wavelengths, of the light in the two or more paths.

Preferably, the apparatus comprises a computer for analysing the measurement of the light in the two or more paths to provide a measure of the optical wavefront. In certain embodiments the apparatus is configured to illuminate the fovea of an eye and capture light reflected or scattered from the fovea, for providing a measure of the power or aberrations of the eye.

According to a fourth aspect of the present invention there is provided a hyperspectral imaging apparatus comprising:
an illuminator for producing a plurality of elongated beams having a wavelength range covering selected spectral reflection or absorption features of an object;
an optical system for projecting said plurality of elongated beams onto a corresponding plurality of areas on said object and for capturing light reflected, scattered or fluoresced from said areas;
a linear aperture array for confocal gating of the captured light reflected, scattered or fluoresced from said areas; and
a spectrometer comprising a two-dimensional sensor array for simultaneous detection, over a range of wavelengths, of the captured light reflected, scattered or fluoresced from said areas.

In certain embodiments the apparatus is configured for hyperspectral imaging of an object comprising the retina of an eye. The optical system is preferably configured to project the plurality of elongated beams towards the eye at angles selected to suppress the capture of specular reflections of the elongated beams from the cornea of the eye. In certain embodiments the optical system comprises a focal plane relay including an aperture for suppressing the capture of specular reflections of the elongated beams from the cornea of the eye. In certain embodiments the optical system comprises a dispersive element for angularly dispersing the plurality of elongated beams, so as to reduce the intensity of the elongated beams on the retina.

The apparatus may comprise a cylindrical lenslet array for focusing the captured light onto the apertures of the linear aperture array. In certain embodiments the apparatus comprises a birefringent element for polarisation-sensitive detection of the captured light.

Preferably, the apparatus comprises a beam splitter for directing elongated beams towards the object and for directing the captured light towards the spectrometer. In certain embodiments the beam splitter is configured to provide a reference beam for coherence-resolved detection of the captured light.

The apparatus may comprise a deflection element for moving the areas on the object in a direction substantially perpendicular to the long axes of the areas, for imaging larger areas of the object.

In certain embodiments the illuminator comprises: an optical source for emitting an elongated stripe of light; and an array of one-dimensional focusing elements for producing the plurality of elongated beams from the elongated stripe of light. The illuminator may comprise a beam redirection element for steering individual beams of the plurality of elongated beams in selected directions. The beam redirection element is preferably positioned at a focal plane of the array of one-dimensional focusing elements. Preferably, the optical source is substantially spatially incoherent. In certain embodiments the illuminator comprises an aperture for adjusting the spatial coherence of the light emitted from the optical source.

According to a fifth aspect of the present invention there is provided an apparatus for hyperspectral confocal imaging of a sub-surface region of an object with enhanced rejection of surface reflected light, said apparatus comprising:
an illuminator for forming a spatially structured illumination field for projection onto a sub-surface region of an object;
an optical power element positioned in the far field of the structured illumination field;
a spatially structured confocal aperture configured to accept light reflected, scattered or fluoresced from said sub-surface region of said object;
a spectrometer comprising a dispersive optical relay for projecting a wavelength dispersed image of the structured confocal aperture onto a two-dimensional sensor array; and
a spatially selective directional coupling element for directing at least a portion of the structured illumination field towards said object and for directing at least a portion of the reflected, scattered or fluoresced light towards said spatially structured confocal aperture, such that the capture of light reflected from a front surface of said object is suppressed.

In certain embodiments the apparatus is configured for hyperspectral confocal imaging of a sub-surface region of an object comprising the retina of an eye or the cornea of an eye.

According to a sixth aspect of the present invention there is provided a method for imaging an eye, said method comprising the steps of:
illuminating, with a multi-wavelength optical beam, a volume of the eye, said volume to be imaged in three spatial dimensions, said volume having an elongated lateral cross-sectional area with a short axis, a long axis and an aspect ratio defined by the ratio of the long axis to the short axis;
capturing and anisotropically transforming light scattered or reflected from the illuminated volume;
splitting light emitted from an optical source into a reference beam and said multi-wavelength optical beam;
combining said reference beam with the captured light;
sampling the anisotropically transformed captured light in one dimension; and
simultaneously capturing, with a measurement system comprising a two-dimensional sensor array, phase and amplitude information over a range of wavelengths of the sampled anisotropically transformed captured light.

According to a seventh aspect of the present invention there is provided a method for optical imaging, said method comprising the steps of:
illuminating, with a multi-wavelength optical beam, a volume of an object, said volume to be imaged in three spatial dimensions, said volume having an elongated lateral cross-sectional area with a short axis, a long axis and an aspect ratio defined by the ratio of the long axis to the short axis;
capturing and anisotropically transforming light scattered or reflected from the illuminated volume;
splitting light emitted from an optical source into a reference beam and said multi-wavelength optical beam;
combining said reference beam with the captured light;
sampling the anisotropically transformed captured light in one dimension; and
simultaneously capturing, with a measurement system comprising a two-dimensional sensor array, phase and amplitude information over a range of wavelengths of the sampled anisotropically transformed captured light.

According to an eighth aspect of the present invention there is provided a method for wavefront sensing, said method comprising the steps of:
illuminating, with a multi-wavelength optical beam, an area of an object;
capturing an optical wavefront comprising light scattered or reflected from the illuminated area;
splitting the captured light into two or more paths having different optical delays;
simultaneously sampling, in one dimension, the captured light in the two or more paths; and
simultaneously measuring over a range of wavelengths, with a measurement system comprising a two-dimensional sensor array, the light in the two or more paths.

According to a ninth aspect of the present invention there is provided a method for hyperspectral imaging, said method comprising the steps of:
producing a plurality of elongated beams having a wavelength range covering selected spectral reflection or absorption features of an object;
projecting said plurality of elongated beams onto a corresponding plurality of areas on said object;
capturing light reflected, scattered or fluoresced from said areas;
confocally gating the captured light reflected, scattered or fluoresced from said areas; and
simultaneously detecting over a range of wavelengths, with a spectrometer comprising a two-dimensional sensor array, the captured light reflected, scattered or fluoresced from said areas.

According to a tenth aspect of the present invention there is provided a method for hyperspectral confocal imaging of a sub-surface region of an object with enhanced rejection of surface reflected light, said method comprising the steps of:
forming a spatially structured illumination field;
projecting, with a system of optics comprising an optical power element positioned in the far field of the structured illumination field, light in said structured illumination field onto said sub-surface region of said object;
accepting, with a spatially structured confocal aperture, light reflected, scattered or fluoresced from said sub-surface region of said object;
projecting a wavelength dispersed image of the confocally accepted light onto a two-dimensional sensor array; and
directing, with a spatially selective directional coupling element, at least a portion of the structured illumination field towards said object and directing at least a portion of the reflected, scattered or fluoresced light towards said spatially structured confocal aperture, such that the capture of light reflected from a front surface of said object is suppressed.

### BRIEF DESCRIPTION OF THE DRAWINGS

Preferred embodiments of the invention will now be described, by way of example only, with reference to the accompanying drawings in which:

FIG. 1 illustrates in schematic oblique view an approach for mapping light reflected or scattered from an elongated illuminated volume of an object onto a 2-D sensor array;

FIG. 2A shows in schematic plan view an optical imaging apparatus for in-vivo tomographic imaging of the retina of a human eye, according to an embodiment of the present invention;

FIG. 2B depicts a portion of the apparatus shown in FIG. 2A in an orthogonal plane;

FIG. 3 shows in schematic oblique view an optical imaging apparatus according to an embodiment of the present invention;

FIG. 4 illustrates in schematic plan view an optical imaging apparatus according to an embodiment of the present invention;

FIG. 5 shows in schematic plan view an apparatus for multi-wavelength wavefront analysis of light reflected or scattered from an object, according to an embodiment of the present invention;

FIG. 6A depicts in schematic plan view an apparatus for in-vivo wide field hyperspectral imaging of the retina of an eye, according to an embodiment of the present invention;

FIG. 6B shows in schematic oblique view the construction of a light source suitable for use in the apparatus of FIG. 6A;

FIG. 6C shows in schematic plan view an alternative beam splitter for use in the apparatus of FIG. 6A;

FIG. 7A depicts in schematic plan view an apparatus for in-vivo wide field hyperspectral imaging of the retina of an eye, according to an embodiment of the present invention; and

FIG. 7B depicts in schematic plan view an apparatus for hyperspectral confocal imaging of a sub-surface region of an object, according to an embodiment of the present invention.

### DETAILED DESCRIPTION OF THE INVENTION

The current invention provides techniques and apparatus for snapshot tomographic imaging across an elongated volume of an object such as the retina of a human eye with resolution that is substantially isotropic in two or three dimensions, with application of digital refocusing in both lateral axes so that larger volumes of the object can be constructed with high resolution by shifting the elongated illuminated area substantially perpendicularly to its long axis. The full-field nature of the acquisition enables registration of phase between individual volume acquisitions. The technique is also suitable for high-resolution hyperspectral analysis or metrology over extended areas or volumes of an object. By selecting or controlling the spatial coherence of the illumination, multiply-scattered light can be suppressed while providing sufficient signal coherence for aberration correction and digital refocusing. In certain embodiments polarisation information can be extracted from an object by knowing or controlling the polarisation state of the illumination and utilising dual polarisation detection.

The cross-sectional area of an object that can be accessed in a single snapshot acquisition using the techniques of the present invention is considerably larger than in the prior art, and may be example be of order 3 mm×50 μm compared to areas of order 100 μm×100 μm in US 2016/0345820 A1. The ability to access larger areas in a single frame reduces the number of frames required to image a given overall area of an object, thereby reducing the total acquisition time. Furthermore with a highly elongated illumination area it may only be necessary to scan the illumination in one dimension rather than two, simplifying the apparatus and the registration of individual snapshot images. In basic terms the techniques of the present invention trade speed for imaging depth or number of resolvable wavelengths, allowing a larger area metric or resolution in a given acquisition time for a given 2-D sensor array. The techniques may be particularly advantageous for retinal imaging, where the features of interest typically occupy a depth range of only a few hundred microns compared to the ˜10 mm depth range required for imaging of the anterior segment of the human eye. In particular, the reduction in the time required to image large areas of the retina will be beneficial for ameliorating the effect of motion artefacts in OCT Angiography.

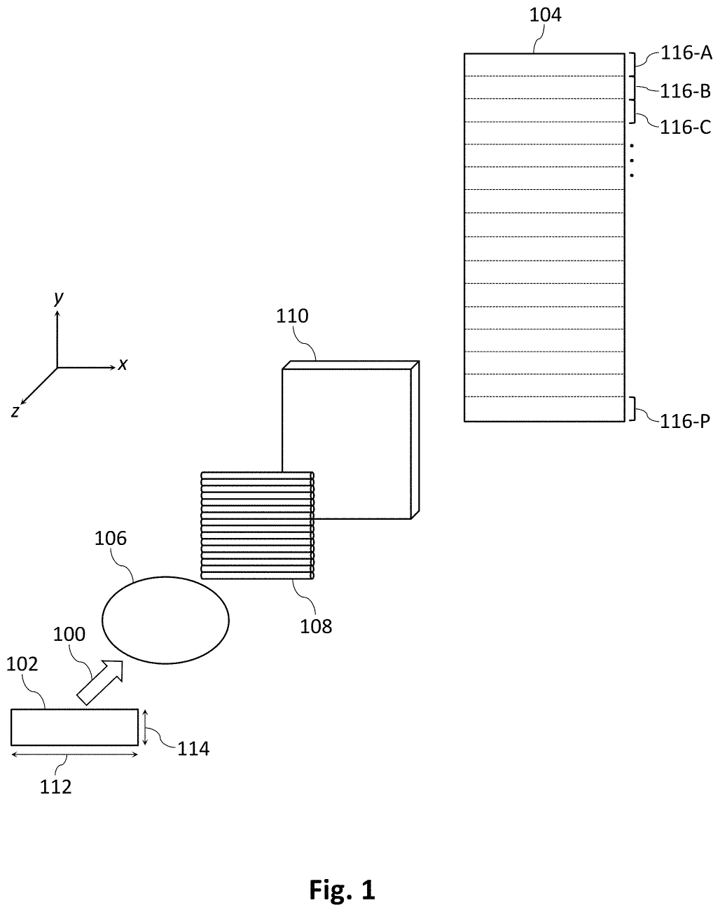

FIG. 1 shows in schematic oblique view an approach for mapping light 100 reflected or scattered from an elongated illuminated volume of an object such as a retina, having a cross-sectional area 102, onto a two-dimensional (2-D) sensor array 104, using an optical system 106 illustrated for simplicity by a single lens, a spatial sampling element in the form of a cylindrical lenslet array 108 and a dispersive relay 110. Spatial sampling may alternatively be provided by a linear aperture array or a combination of a lenslet array and an aperture array. As explained below, a suitable dispersive relay comprises a combination of lenses and a wavelength dispersive element such as a grating. The illuminated area 102, shown here as rectangular although it may be any regular or irregular elongated shape, has an aspect ratio defined by the ratio of a long axis 112 to a short axis 114. Depending on the details of the illumination source and optics, the aspect ratio of the illuminated area 102 may for example be at least ten, or at least twenty, or at least fifty, or at least one hundred.

In one specific example the illuminated area 102 is 9 mm×48 μm, corresponding to an aspect ratio of 187.5, and the 2-D sensor array 104 is a 3000×4000 (12 Mpixel) array with 4 μm square pixels. It is relatively straightforward to image the long axis 112 of the illuminated area 102 onto the corresponding (x) axis of the sensor array 104, with dispersion along the spectral (y) axis as in conventional line field OCT, to provide a lateral resolution of 3 μm in the x-axis (9 mm over 3000 pixels). However it is more challenging to obtain a snapshot C-scan with comparable resolution in the short axis 114 of the illuminated area 102. In several embodiments described in detail below, a cylindrical lenslet array 108 samples the reflected or scattered light 100 in a dimension substantially parallel to the short axis 114, with the optical system 106 configured to capture and anisotropically transform the reflected or scattered light 100. In preferred embodiments the anisotropic transformation acts to reduce the aspect ratio of the captured light field such that its aspect ratio at the cylindrical lenslet array 108 is less than the aspect ratio of the elongated illuminated area 102.

In certain embodiments the cylindrical lenslet array 108 is positioned at or near an image plane of the optical system 106, for image plane sampling of the reflected or scattered light 100 in one dimension, or more precisely at multiple positions in one dimension, in this case the y-axis. The dispersive relay 110 then disperses the segmented image onto separate sections of the 2-D sensor array 104. In the illustrated example a sixteen-element cylindrical lenslet array 108 samples the image in the y-axis for dispersion and projection onto separate sections 116-A, 116-B, 116-C . . . 116-P of the sensor array 104 as shown, effectively providing simultaneous acquisition of sixteen B-scans at the expense of a sixteen-fold reduction in depth range because of the reduced number of wavelengths per section 116 of the sensor array. However this image plane sampling approach requires the optical system 106 to have high magnification in the y-axis, which may be problematic in some applications, particularly where the cornea and intra-ocular lens of the eye contribute to the imaging of the reflected or scattered light 100.

Surprisingly, we have found that high lateral resolution in the short axis 114 of the illuminated area 102 can be achieved for a spectral domain snapshot volume OCT imaging system, without requiring highly anisotropic image magnifying optics, by sampling the short axis 114 in the Fourier plane rather than in the image plane. In this case the cylindrical microlens array 108 is placed at or near a Fourier plane of the optical system 106, converting lateral position along the short axis 114 to angle such that each section 116-A, 116-B . . . 116-P of the sensor array 104 receives information from across the short axis. That is, with Fourier plane sampling there is no 1:1 correspondence between the sections 116-A, 116-B . . . 116-P of the sensor array 104 and segments of the reflected or scattered light 100, unlike the case with image plane sampling.

Irrespective of whether the y-axis sampling is in the Fourier plane or the image plane, sampling of the reflected or scattered light 100 in the x-axis is provided by the pixels of the sensor array 104. This x-axis sampling may be in the Fourier plane or the image plane, depending among other things on the configuration of the optical system 106. For efficient use of the 2-D sensor array 104 the optical system 106 and cylindrical lenslet array 108 are preferably configured such that the reflected or scattered light 100 is projected onto a substantial portion of the 2-D sensor array, generally with some margin for relaxed alignment tolerance. High-speed 2-D sensors with a large pixel count, e.g. multi-Mpixel sensors, enable capture of a large amount of data in a single frame, either with a global shutter or a rolling shutter. In situations where acquisition speed is paramount it is often desirable to restrict the field of the sensor to achieve faster frame rates. The present application on the other hand takes advantage of simultaneous acquisition, so to maximise the amount of data captured in a single frame it is preferable to utilise a substantial portion of the 2-D sensor array, i.e. to have a high pixel count in both dimensions. In state of the art visible or near IR light sensors this could mean projection onto at least 2000 pixels per dimension, but in any case it is expected that for various types of 2-D sensors more than 100 pixels per dimension would be illuminated.

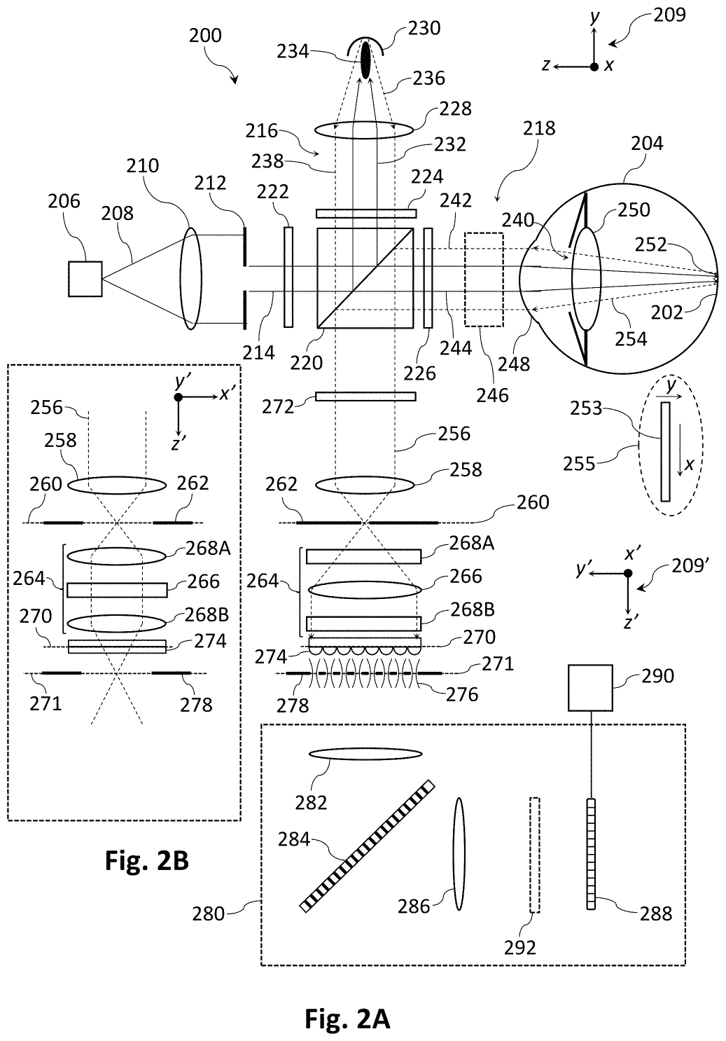

FIG. 2A shows in schematic plan view an optical imaging apparatus 200 for in-vivo tomographic imaging of the retina 202 of a human eye 204, according to an embodiment of the present invention. The apparatus 200 comprises a broadband line source 206 emitting a multi-wavelength beam of light 208 elongated in the x-axis, i.e. into the page, as defined by the Cartesian axes 209. In certain embodiments the line source 206 is a spatially coherent source, e.g. a single spatial mode source such as an optical fibre-based supercontinuum source or a superluminescent light emitting diode (SLED) with appropriate anamorphic or astigmatic lensing. Preferably however the source 206 is a partially spatially incoherent source such as a high-power semiconductor gain medium having a stripe configuration, where feedback has been suppressed by antireflection coatings or angled facets allowing the gain medium to be driven below lasing threshold to produce broadband or partially incoherent light. The use of low coherence sources is generally advantageous for safety in in-vivo ocular examination because the emitted light cannot be focused to a small high-intensity spot on the retina. Other suitable partially spatially incoherent line sources include linear arrays of SLEDs, LEDs or wavelength-broadened VCSELs or laser diodes. In some embodiments a partially spatially incoherent source is provided by scanning one or more spatially coherent sources, such as an array of SLEDs, along the x-axis to create an elongated source. Within a single acquisition frame of a 2-D sensor array 288 the temporal incoherence between the different source positions achieves a degree of virtual spatial incoherence. Advantageously, this source scanning scheme allows a longer exposure time without fringe washout, and hence the use of lower peak power sources for the same average power.

In yet other embodiments the line source 206 is fully spatially incoherent, derived for example from laser-driven plasma, fluorescent or phosphorescent sources that may be confined within a single mode planar waveguide and pumped by another light source. When the object being imaged is an eye 204 as shown, the apparatus 200 will typically have a source 206 emitting light in the visible or near infrared regions of the electromagnetic spectrum. However this is not a limitation of the present invention and in general light sources emitting in the ultraviolet, visible or infrared regions of the electromagnetic spectrum may be used depending on the application. An etalon may be included to extend the coherence length of the light emitted from the line source 206.

The elongated beam 208 emitted from the source 206 is collimated by a spherical lens 210 with focal length of, say, 20 mm, then passed through an optional aperture 212 that restricts the numerical aperture (NA) of the lens 210 and hence the minimum diffraction limited spot size that can be created if the apertured beam 214 is subsequently focused with a lens of a given focal length. The size of the aperture 212 can also be chosen to adjust the spatial coherence of the beam 208 emitted from the source 206, with a smaller aperture generally increasing the spatial coherence of the transmitted light 214. In certain embodiments the aperture 212 is circular, while in other embodiments the aperture is rectangular to restrict the beam 214 to different extents in the x- and y-axes.

The apertured source beam 214 is split into a reference path 216 and a sample path 218 with a beam splitter 220. In the illustrated embodiment the beam splitter 220 is a polarisation beam splitting cube that splits the apertured source beam 214 according to the relative angle of the beam's polarisation state, which may be set by a polarising element 222. A power beam splitter may be used instead, although the combination of a polarisation beam splitter 220 and quarter wave plates 224, 226 is generally less wasteful of source light.

The reference path 216 comprises an optical power element 228 in the form of a lens and a curved reflective element 230. The optical power element 228 focuses the collimated beam 232 to a waist 234 that may for example be 100 μm by 20 mm. The curved reflective element 230 may be a short focal length cylindrical mirror that transforms the beam waist 234 in the short axis to be an angularly divergent beam 236. This divergent beam is collimated by the lens 228 to form a reference beam 238 of comparable dimension to a dilated pupil 240, suitable for mixing with the returning sample beam 242 as described below. For applications where shaping of the reference beam 238 is not required, the lens 228 and curved reflective element 230 may be replaced by a planar mirror.

Multi-wavelength light 244 in the sample path 218 is directed to the eye 204, preferably by a 4F lens relay system 246 which may have focusing and angular beam steering capabilities as described for example in published US patent application No US 2019/0223729 A1 entitled ‘Apparatus and method for confocal microscopy using dispersed structured illumination’, the contents of which are incorporated herein by reference. The combined focusing power of the cornea 248 and intra-ocular lens 250 transforms the multi-wavelength sample beam 244 to a laterally elongated beam waist that illuminates a volume 252 of the retina 202, with an elongated cross-sectional area 253 as shown in the inset view 255. The illuminated area 253 on the retina, i.e. the beam waist dimensions, will generally be of order tens of microns by several millimetres in the y- and x-directions respectively, for example 50 μm×3 mm corresponding to an aspect ratio of sixty. The illuminated area 253 may be substantially rectangular or elliptical in shape, or any other elongated shape with a longer dimension significantly larger than a shorter dimension. In preferred embodiments with a partially spatially incoherent line source 206 the light at the beam waist is relatively coherent across the shorter dimension, but coherent across only a small distance, e.g. 50 μm, in the longer dimension. On the other hand if the light is derived from a single spatial mode source 206 the projection onto the retina 202 will be coherent in both axes but will still have a highly elongated shape, e.g. 50 μm×3 mm. Noting again the influence of the aperture 212 on the spatial coherence of the sample beam 244, it is preferable for there to be some coherence across the long axis of the illuminated area 253 to allow digital refocusing.

Light 254 will be reflected or scattered from the illuminated volume 252 of the retina 202 in a large range of angles and can be captured within an angular range determined by the size of the dilated pupil 240, with the larger numerical aperture components enabling higher resolution imaging of the retina 202. The reflected or scattered light 254 will be collimated, at least for the case of an emmetropic eye, by the intra-ocular lens 250 and cornea 248 to form a returning sample beam 242. A polarisation component of this returning sample beam is reflected by the polarisation beam splitter 220, forming a return beam 256 with the substantially overlapping reference beam 238. At this point the returning sample beam 242 and reference beam are orthogonally polarised so will not interfere until they are analysed by a polariser 272. Since the reflectivity of the retina 202 will generally be significantly less than that of the reference arm mirror 230, it is usually advantageous for the polariser 272 to be oriented to pass a large fraction of the returning sample beam 242 and a smaller fraction of the reference beam 238. If on the other hand the sample and reference paths 218, 216 are split and recombined with a power beam splitter the returning sample beam 242 and reference beam 238 will interfere as soon as they are recombined.

A spherical lens 258 images the illuminated area 253 of the retina 202 to an image plane 260 where there is positioned a linear, i.e. slit-shaped, aperture 262 for excluding light which has been multiply-scattered or is from the edges of the illuminated volume 252.

Light passing through the aperture 262 proceeds to an anisotropic relay system 264 of cylindrical lenses, the operation of which is described with reference to a set of Cartesian axes 209′ rotated with respect to the Cartesian axes 209 to account for the redirection at the beam splitter 220. For ease of explanation the relay system 264 and surrounding optics are also illustrated in the orthogonal (x′z′) plane in FIG. 2B. The relay system 264 comprises a first cylindrical lens 266 of focal length 2f for focusing in a first axis (the y′-axis) and a pair of cylindrical lenses 268A, 268B of focal length f for focusing in a second axis (the x′-axis). The first cylindrical lens 266 is positioned at a distance from the aperture 262 equal to its focal length 2f and acts to collimate the return beam 256 in the first axis, i.e. the short axis of the aperture 262. The pair of cylindrical lenses 268A, 268B act to collimate then refocus the light from the aperture 262 in the second axis, i.e. the long axis of the aperture 262. The first cylindrical lens 266 therefore forms a 2F relay system in the first axis while the cylindrical lenses 268A, 268B form a 4F relay system in the second axis, such that the plane 270 is a Fourier plane for light in the first axis and the plane 271 is an image plane for light in the second axis. The plane 271 is preferably displaced from the plane 270 by an optical equivalent distance determined by approximately twice the focal length of the lenslets in a cylindrical lenslet array 274 placed at or near the plane 270.

The cylindrical lenslet array 274 forms a spatial sampling element for sampling the return beam 256 at multiple positions in the first (y′) axis, i.e. the axis for which the plane 270 is a Fourier plane, effectively sampling the return beam from different parts of the far field. The resulting array of focused lines 276 pass through an array of linear apertures 278 placed at or near the plane 271 before proceeding to a measurement system 280 for simultaneous measurement of phase and amplitude information over a range of wavelengths of the captured return beam 256 sampled by the cylindrical lenslet array 274. By selecting or controlling the spatial coherence of the illumination, multiply-scattered light can be suppressed while providing sufficient signal coherence for aberration correction and digital refocusing. This scheme is advantageous in that it is able to physically separate the wavefront measurements of numerous sample regions that are each illuminated by light that is not spatially coherent to generate a full volume SD-OCT image while avoiding the noise penalties and sensitivity loss that would occur if the incoherent signals were captured overlapping on the same set of pixels. The measurement system 280 comprises a collimating lens 282, a dispersive element 284 and a focusing lens 286 that creates dispersed line arrays for detection at a 2-D sensor array 288. In the illustrated embodiment the dispersive element 284 is a transmissive grating, but in other embodiments it may for example be a prism, a reflective grating or a combination of a prism and a grating.

The relay system 264 of cylindrical lenses and the spherical lens 258 form an optical system configured to cooperate, when in use, with the optical power elements 248, 250 of the eye 204 for capturing and anisotropically transforming light 254 reflected or scattered from the illuminated volume 252 of the retina 202. It will be appreciated that this optical system could be reconfigured for imaging other structures in the eye 204, such as the intraocular lens 250 or the cornea 248. Preferably, the anisotropic transformation serves to increase the spatial extent of the captured light in the first axis, corresponding to the short axis of the illuminated area 253, relative to the second axis, so that the aspect ratio of the captured light at the cylindrical lenslet array 274 is less than the aspect ratio of the illuminated area 253. In the illustrated embodiment the spatial extent of the captured light in the first (short) axis is determined by appropriate choices of the focal lengths of the spherical lens 258 and cylindrical lens 266. In the illustrated example the far field at the dilated pupil 240 is expanded by a factor of two onto the cylindrical lenslet array 274, e.g. from 8 mm to 16 mm, by choosing the focal length of the spherical lens 258 to be half that of the cylindrical lens 266. It should be noted however that in this embodiment an anisotropic transformation of the reflected or scattered light is ensured by the fact that the reflected or scattered light is sampled in the Fourier plane in one axis and in the image plane in a second axis, by the cylindrical lenslet array 274 and the pixels of the 2-D sensor array 288 respectively.

In one particular embodiment the 2-D sensor array 288 is a high-speed Flash 4K 4000×1000 pixel sensor from Teledyne Technologies, Inc, capable of operating at 1800 frames per second (fps). With a 16-element cylindrical lenslet array 274 sampling the return beam 256 in the Fourier plane, and with 200 wavelengths per sample for dispersion along the 4000-pixel axis of the sensor array 288, the apparatus 200 is able to provide the equivalent of 16×1000×1800 A-scans per second. This equates to 28.8M A-scans per second, which is approximately 100 times faster than the fastest currently available spectral domain OCT systems, which offer around 250k to 300k A-scans/sec. In another embodiment the 2-D sensor array 288 is a 12 Mpixel (4000×3000) sensor providing 3000×16=48k A-scan equivalent resolution per frame, so that even a moderately low speed sensor, with a frame rate of say 60 Hz, could provide nearly 3M A-scans per second, enabling low-cost but high-speed retinal imaging.

Phase and amplitude data acquired with the 2-D sensor array 288 are processed with a computer 290 equipped with suitable machine-readable code to recover the retinal image at high resolution over a range of depths, e.g. with an on-retina pixel size of approximately 3×3 microns. Even over a small range of axial depths the line scan images will often become defocused along their length, necessitating some form of corrective processing such as digital refocusing.

Noting that the light 254 reflected or scattered from the retina 202 has been sampled in the Fourier plane in a first axis and in the image plane in a second axis, by the cylindrical lenslet array 274 and the pixels of the 2-D sensor array 288 respectively, one approach to processing the data will be described with reference to FIG. 1.

In a first step, phase and amplitude data from each of the sections 116-A . . . 116-P of the 2-D sensor array 104, which may for example have 200 resolvable wavelengths in the dispersive (y) axis, are Fourier transformed in the dispersive axis to transform from wavelength to axial depth, then Fourier transformed in the x-axis so that the data in both axes is now in the Fourier domain, i.e. the optical far field. We now have the complete volume 3-D Fourier information. In a second step the data is convolved with a focusing or aberration function, which may be termed a ‘digital adaptive optics function’, or ‘DAO function’, that can be determined iteratively for each depth to be visualised. In certain embodiments the choice of focusing or aberration function is determined analytically or iteratively by using an image focus metric or ‘guide star’ as may be provided by the reflections off individual cones in the retina. Finally, for each depth and choice of DAO function the 3-D data set is Fourier transformed to recreate the desired focal plane as is well known in the art. In general the resulting image will be a representation of an optical characteristic of the retina, such as phase, reflectivity, refractive index, refractive index changes and attenuation over the illuminated volume 252. If the apparatus 200 is configured for polarisation diverse detection, e.g. with the addition of a polarisation separation element such as a walk-off plate 292 between the focusing lens 286 and the 2-D sensor array 288, the optical characteristic may be birefringence or degree of polarisation.

To image larger regions of the retina 202, beam steering optics in the lens relay system 246 can move the illuminated volume 252 to one or more adjacent or nearby locations, preferably with some overlap for co-registration of acquired data, and preferably in a direction substantially perpendicular to the long axis of the illuminated area 253, i.e. in a direction substantially parallel to the y-axis. A tomographic image of an extended volume of the retina 202 can thereby be constructed from the series of snapshot acquisitions in an approach similar to the line field holoscopy technique described in published US patent application No 2014/0028974 A1, but with the significant advantage that high resolution is maintained in both lateral axes. Consequently the full volume will have high resolution in both axes even in the presence of lateral or axial shifts between adjacent snapshot acquisitions. Preferably the apparatus 200 is configured so that the resolution is substantially isotropic in both lateral axes.

For OCT-A applications the apparatus 200 can be configured for capture of phase and amplitude information in two or more sequential frames of the sensor array 288 at a given location on the retina 202, optionally with pulsing of the source 206, for processing in the computer 290 to allow measurement of changes due to blood flow in the illuminated volume 252. A frame rate of 300 Hz for example would provide a 3.33 ms delay between frames, suitable for robust measurement of changes due to blood flow, whereas the eye motion will be small enough for there to be a significant overlap volume between the sequence of frames to allow co-registration and subtraction of the bulk eye motion. The process can be repeated at additional locations on the retina 202, preferably with partial overlap for registration, for measuring blood flow in larger portions of the retina. In other embodiments the motion in the y-axis of the illumination between each frame can be small compared to the y-dimension of the illuminating beam such that an overlap region of the reconstructed volume can be used for determining the blood flow in a volume or projection image of the retina for example.

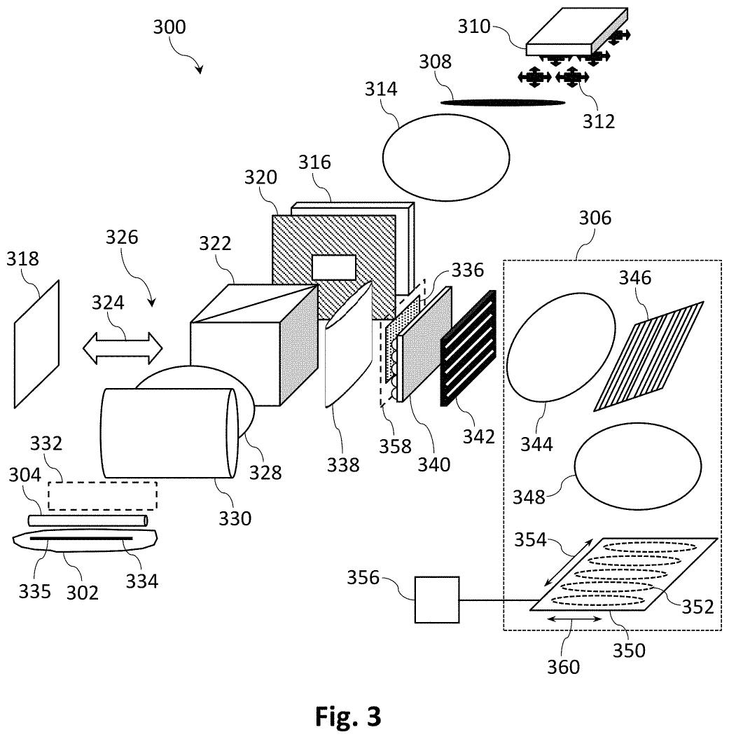

FIG. 3 shows in schematic oblique view an optical imaging apparatus 300 for imaging an object 302, which may for example be the cornea of an eye or a non-ocular biological sample, according to another embodiment of the present invention. In this embodiment an anisotropic magnification of the focal plane of a cylindrical objective lens 304 onto a 2-D spectrometer 306 enables a cylindrical lenslet-based optical system to image a contiguous volume of the object 302. A highly asymmetric beam 308 of spatially incoherent and partially spectrally incoherent light is generated from a thin plate 310 of a phosphor crystal such as Ce-YAG pumped by an array of blue LEDs 312. Incoherent light from the phosphor ions is collimated by an optical power element 314, shown here as a lens but which could equally be a concave mirror, and the resultant beam passed through an optional Fabry-Perot etalon 316 to extend the coherence length of the individual spectral components. In one particular example a Fabry-Perot etalon 316 with a free spectral range of 200 GHz and a finesse of 20 may be chosen to yield a bandwidth of approximately 10 GHz in each spectral bin to provide improved tolerance for length matching the coherent interference provided by a reference mirror 318. An aperture 320 may be included to provide a desired level of spatial coherence at the object 302, with a smaller aperture providing greater spatial coherence, and hence range of digital refocusing, at the expense of reduced intensity.

A portion of the apertured light is reflected by a beam splitter 322 to form a reference beam 324, which is reflected by a mirror 318. This embodiment utilises a power beam splitter 322, although a polarisation beam splitter could be used instead. Additional elements or features may be included in the reference arm 326, e.g. shaping of the reference mirror 318 to ensure optimal overlap with the returning sample light when being redirected towards the spectrometer 306. Source light that passes undeflected through the beam splitter 322 is directed to a combination of a spherical lens 328 and a relatively weak cylindrical lens 330 to provide focusing in one axis at a focal plane 332 of a short focal length cylindrical lens 304 such as a rod lens, illuminating a volume 334 of the object 302 having a highly elongated cross-sectional area 335. In one particular example the combination of a spherical lens 328, cylindrical lens 330 and rod lens 304 with respective focal lengths of 50 mm, 500 mm and 2 mm produces an illuminated area 335 of 200 μm×12 mm on the object 302, corresponding to an aspect ratio of sixty.

Light reflected or scattered from the illuminated volume 334 of the object 302 is captured and anisotropically transformed with an optical system comprising a cylindrical lens 338 in addition to the lenses 304, 330 and 328, and relayed via the beam splitter 322 where it mixes with the reference beam 324. In this embodiment this optical system magnifies the captured light to different extents in the short and long axes of the illuminated area 335, with the cylindrical lens 338 and the spherical lens 328 providing magnification in the long axis of the illuminated area 335 and the magnification in the orthogonal short axis provided by the ratio of the focal length of the combined lens 328, 330 to the focal length of the cylindrical objective lens 304. In one particular example a 25× magnification can be provided by a focal length ratio of 50 mm to 2 mm, enabling a 200 μm wide short axis to be magnified to a 5 mm wide image 336 that can be sampled with a spatial sampling element in the form of a cylindrical lenslet array 340 and a linear aperture array 342. Preferably the apparatus is configured such that the lenslet array 340 is at or near the short axis image plane 358, while the aperture array 342 is at or near the long axis image plane.

Light passed by the aperture array 342 proceeds to a 2-D spectrometer 306 comprising a collimating lens 344, a grating 346, a focusing lens 348 and a 2-D sensor array 350, for simultaneous measurement of phase and amplitude information over a range of wavelengths. Portions of the short axis image 336 sampled by the cylindrical lenslet array 340 are dispersed onto separate portions 352 of the 2-D sensor array 350 separated in the dispersive axis 354, with columns of pixels in the orthogonal direction 360 providing lateral resolution in the long axis of the illuminated area 335. In one particular example, with a 12 Mpixel (4000×3000) sensor array 350, the magnified image 336 of the 200 μm wide short axis is sampled with a forty-element cylindrical microlens array 340 to provide a lateral resolution of 5 μm in that axis, while the 12 mm long axis is projected across 3000 pixels of the 2-D sensor array 350 to provide a lateral resolution of 4 μm in the long axis of the illuminated area 335.

Phase and amplitude data acquired with the 2-D sensor array 350 can then be processed with a computer 356 equipped with suitable machine-readable code to recover depth-resolved information with lateral resolution in two axes across the illuminated volume 334. The processing differs from that described for the apparatus 200 of FIG. 2A because the holoscopy information is now in the image plane in both axes, with lines from the cylindrical lenslets 340 corresponding to spatial pixels in the short axis of the illuminated area 335. In one approach the data set read out by the computer 356 is Fourier transformed and relevant aberration or focus corrections applied to provide a high-resolution image throughout the whole illuminated volume 334. This scheme is likely to be advantageous for objects where the imaging depth requirement, and hence the number of wavelengths required, is quite limited, so the anisotropic imaging ratio is not too high for a practical optical design without overly large focal length requirements, advantageous for a compact apparatus.

The apparatus 300 may be advantageously applied to scanning applications where the apparatus is translated relative to the object 302 by moving the apparatus or the object. For example handheld instruments for scanning of skin for assessing burns or skin conditions such as melanoma can benefit hugely from the high-speed acquisition of volume information with post-processing of the focusing. Because of the reduced number of wavelengths that need to be captured per sampling point, the apparatus can be sufficiently compact for handheld use. It is in fact the digital refocusing that provides the opportunity to use a smaller number of wavelengths, as the apparatus does not rely solely on the coherence gate to provide depth resolution. Instead, the coherence gate is used in combination with digital refocusing to extend the range of imaging depth beyond the Nyquist range that would otherwise be offered by a limited set of wavelengths, with the extended depth range related to the extended coherence length provided by the etalon 316. We note that the wavelength filtering provided by the etalon will generally be non-uniform across the illuminated volume 334 because of the range of emission angles from the phosphor ions in the light source 310, however this can be calibrated across the beam to ensure accurate scaling of the axial depth.

In an alternative embodiment the cylindrical lens 338 is omitted, in which case the pixels of the sensor array 350 will sample the reflected or scattered light in the far field rather than the near field. This situation, where the sampling is in the near field (image plane sampling) in the short axis and in the far field (Fourier plane sampling) in the orthogonal long axis, is similar to the situation depicted in FIG. 2A, except the axis of focusing and the Fourier plane have been swapped relative to the dispersive axis 354.

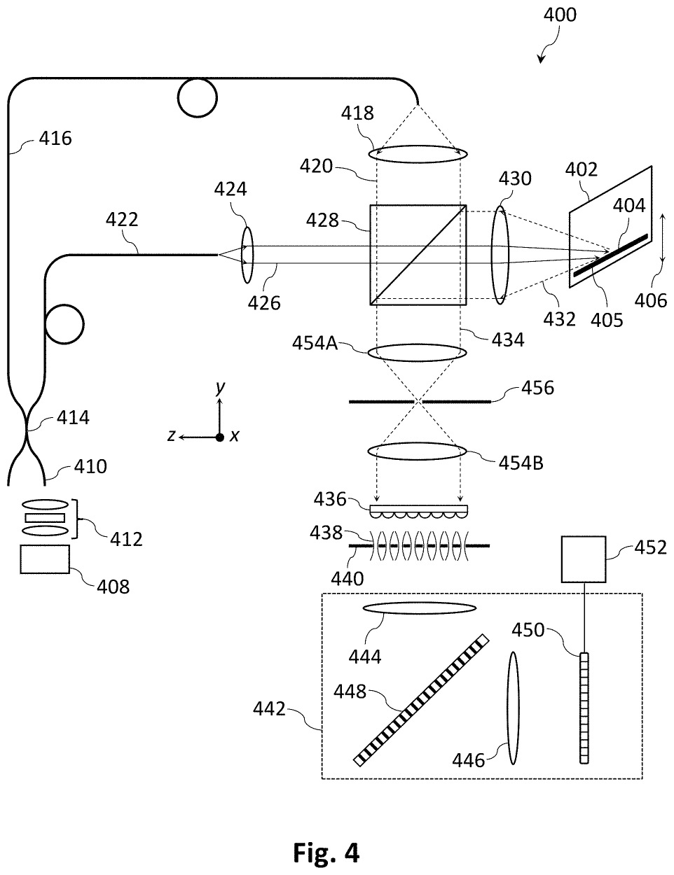

FIG. 4 shows in schematic plan view an optical imaging apparatus 400 according to another embodiment of the present invention, in which relatively large areas or volumes of an object 402 such as a cornea or a non-ocular biological sample can be imaged by scanning an elongated illuminated area 405 in a direction 406 substantially parallel to the short axis of the area 405. The scanning may for example be effected by lateral translation of the object or by angular scanning of a beam-steering mirror. In this embodiment a cylindrical lenslet array 436 provides far field sampling of reflected or scattered light 432 in a direction substantially parallel to the short axis of the illuminated area 405, with dense far field sampling in a direction substantially parallel to the long axis provided by the pixels of a 2-D sensor array 450. In preferred embodiments the aspect ratio of the illuminated area 405 is at least 10:1. The ability to image relatively large volumes of an object 402 with scanning in only one axis, rather than in two axes as in the substantially symmetrical sampling techniques disclosed in the above-mentioned US 2016/0345820 A1, simplifies the optics, which is advantageous for low cost and handheld applications.

The apparatus 400 utilises a spatially coherent but spectrally incoherent light source 408 such as a supercontinuum source coupled to an optical fibre 410. A fibre-coupled source is advantageous for handheld applications because it can be located remotely from the remainder of the instrument. In the illustrated embodiment a fibre-coupled etalon filter 412 is included to pass a comb of wavelengths within a wavelength range of the light source 408, so as to extend the coherence length for each of the wavelengths in the comb. A fraction of the light from the source 408 is split off by a 2×2 fibre coupler 414 into a reference arm 416 and collimated with a spherical lens 418 to provide a collimated reference beam 420. The remainder of the source light is directed into a sample arm 422 and collimated in the y-axis by a cylindrical lens 424 to provide a sample beam 426 incident on a beam splitter 428, depicted here as a power beam splitter although a polarisation beam splitter may be used. The sample light transmitted through the beam splitter 428 is focused in the y-axis and collimated in the x-axis by action of a spherical lens 430 to illuminate a volume 404 of the object 402 having a highly elongated cross-sectional area 405. For clarity the object 402 and the elongated illuminated area 405 are depicted in oblique view, with the short and long axes of the illuminated area parallel to the y- and x-axes respectively.

The dimensions of the illuminated area 405 are determined by the numerical aperture of the optical fibre 410 and the focal lengths of the spherical lens 430 and cylindrical lens 424. For example the combination of an optical fibre 410 of numerical aperture 0.1, a cylindrical lens 424 of focal length 2 mm and a spherical lens 430 of focal length 50 mm will provide an illuminated area 405 of approximately 200 μm×10 mm, corresponding to an aspect ratio of fifty. In certain embodiments one or more apertures are included in the optical train to create a sharp rectangular illumination profile rather than the Gaussian profile of a singlemode optical fibre.

While the elongated illumination profile is incident on the object 402 with a relatively low numerical aperture, light 432 is reflected or scattered from the illuminated volume 404 over a much larger cone of angles. The reflected/scattered spherical wavefronts are collimated by the lens 430 and reflected at the beam splitter 428 where they interfere with the reference beam 420. The interference pattern 434 proceeds via a lens relay comprising a pair of spherical lenses 454A, 454B and a linear aperture 456 to a spatial sampling element in the form of a cylindrical lenslet array 436, where it is sampled in the far field in one axis, i.e. the short axis of the illuminated area 405, based on the pitch of the lenslet array 436. The lens relay 454A, 454B, in combination with the lens 430, forms an optical system for capturing and anisotropically transforming the light 432 reflected or scattered from the illuminated volume 404.

The linear beamlets 438 produced by the cylindrical lenslet array 436 are projected onto a linear aperture array 440 for rejection of light from angles not corresponding to the illuminated volume 404, e.g. multi-scattered light, to provide a well-defined image for analysis in a spectrometer 442, for obtaining phase and amplitude information over a range of wavelengths. As in the previous embodiments the spectrometer comprises lens relay elements 444, 446 and a wavelength dispersive element 448 shown here as a grating. In other embodiments the dispersive and optical power elements can be combined, e.g. in the form of a chirped grating where the variation in grating spacing can provide the optical power for focusing. The wavelength dispersed re-imaging of the aperture array plane onto a 2-D sensor array 450 now provides a Fourier plane interference for each of the different wavelengths, for analysis in a computer 452 using methods described for the Fourier plane sampling embodiments in US 2016/0345820 A1. The use of an etalon 412 to extend the coherence depth offers the opportunity for digital refocusing beyond the Nyquist range. When refocused, the light scattered/reflected from the elongated illuminated volume 404 can be processed into a volume acquired in a single shot, for registration with subsequent volumes acquired with relative motion between the object 402 and the illuminated area 405. Preferably there is some overlap between adjacent volumes to facilitate the registration.

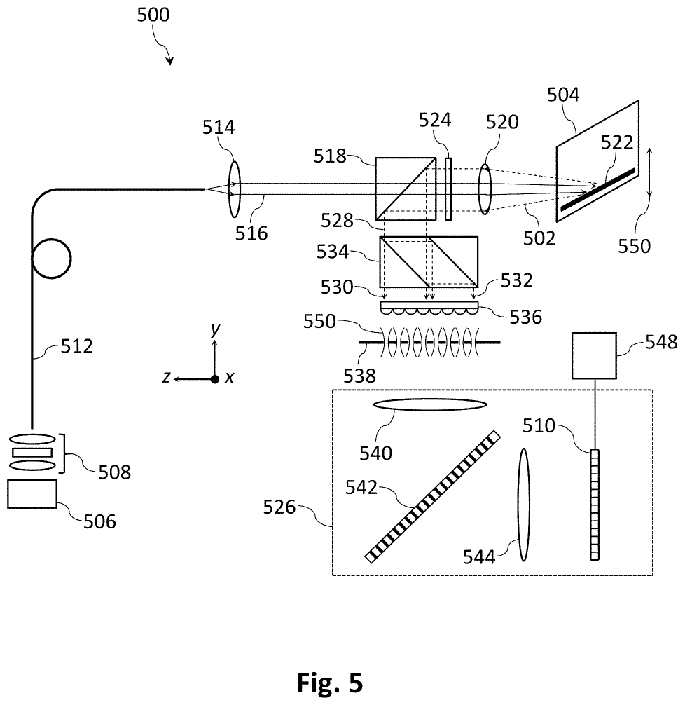

FIG. 5 shows in schematic plan view an apparatus 500 for multi-wavelength wavefront sensing or analysis of light 502 reflected or scattered from an object 504, according to another embodiment of the present invention. This apparatus does not require a reference beam or a spectrally coherent source and is in some ways similar to that depicted in FIG. 4 but without a reference arm. This approach, in which an object 504 is illuminated by a partially incoherent light source 506 and information obtained in the Fourier field, i.e. the far field, has several applications including shear wave interferometry and speckle propagation wavefront sensing. The ability to sample the far field at multiple wavelengths is useful in increasing the speckle contrast, which requires some degree of spectral coherence, and for providing wavelength-dependent information that can be used for tomography or precise determination of a scattering point for metrology. The degree of spectral coherence can be enhanced with wavelength filtering.

The apparatus 500 of FIG. 5 is described with respect to speckle tracking of the far field at two different propagation distances, an application that can be thought of as a multi-wavelength generalisation of a technique disclosed in S. Bonaque-Gonzalez et al ‘Extremely high resolution ocular aberrometry up to 2.4 million points’, Investigative Ophthalmology & Visual Science 60, 603 (July 2019). Light from a broadband source such as a SLED 506 has its wavelength range restricted with a band-pass filter 508 to ensure that the spectra do not overlap on a 2-D sensor array 510, then is coupled into an optical fibre 512 and collimated in the y-axis with a cylindrical lens 514 to provide a multi-wavelength probe beam 516 diverging in the x-axis. At least a portion of the probe beam 516 is transmitted by a polarisation beam splitter 518, then focused in the y-axis and collimated in the x-axis by a spherical lens 520 to illuminate an elongated area 522 of an object 504. It should be noted however that for this speckle tracking application the shape of the illuminated area 522 may be unimportant if the wavefront is not being used to construct an image with near-isotropic resolution, and the cylindrical lens 514 may for example be replaced by a spherical lens to provide a more symmetrical illuminated area. The double pass through a quarter waveplate 524 allows the back-scattered or reflected light 502 to be redirected by the polarisation beam splitter 518 towards a spectrometer 526. In this embodiment the captured optical field 528 reflected or scattered from the illuminated area 522, which is in the Fourier plane due to the collimation by the lens 520, is split into a direct path 530 and a delayed path 532 by a splitting element 534. Light in both paths 530, 532 is sampled in the Fourier plane in one axis, i.e. the short axis of the illuminated area 522, by a spatial sampling element in the form of a cylindrical lenslet array 536 and the resulting linear beamlets 550 passed through a linear aperture array 538 before being measured simultaneously over a range of wavelengths by a spectrometer 526 with components 540, 542, 544 and 510 identical to those employed in the apparatus 400 of FIG. 4. In the illustrated embodiment the splitting element 534 is in the form of a compound beam splitting prism, which could be extended to provide additional paths with increasingly large delays with respect to the direct path 530. In alternative embodiments the splitting element 534 may be a shearing interferometer or other self-interferometric system. The direct and delayed path measurements for each wavelength are processed by a computer 548 equipped with suitable machine-readable code, with the simultaneous capture of the data ensuring that phase relationships between light in the two paths are preserved. As in FIG. 4 extended portions of the object 504 can be acquired by scanning the elongated illuminated area 522 relative to the object, e.g. in a direction 550 substantially parallel to the short axis of the illuminated area 522. The computer 548 may for example process the direct and delayed path measurements to provide a measure of an optical wavefront formed by the light 502 scattered or reflected from the illuminated area 522. The apparatus 500 may for example be configured as an aberrometer for illuminating and capturing light reflected or scattered from the fovea of an eye, for providing a measure of the power or aberrations of the eye.

The apparatus 500 shown in FIG. 5 includes a spherical lens 520 for focusing the illumination onto the object 504 and for collecting a portion of the reflected or scattered light 502. Comparison with FIG. 2A shows that this lens is generally not required if the apparatus is being used to inspect the retina 202 or fovea of an eye 204, unless the eye's optical power elements 248, 250 are unable to provide the requisite imaging due to aberration or non-emmetropia. Similarly, the spherical lens 430 in the apparatus 400 depicted in FIG. 4 may not be required if the apparatus 400 is being used to inspect a retina.

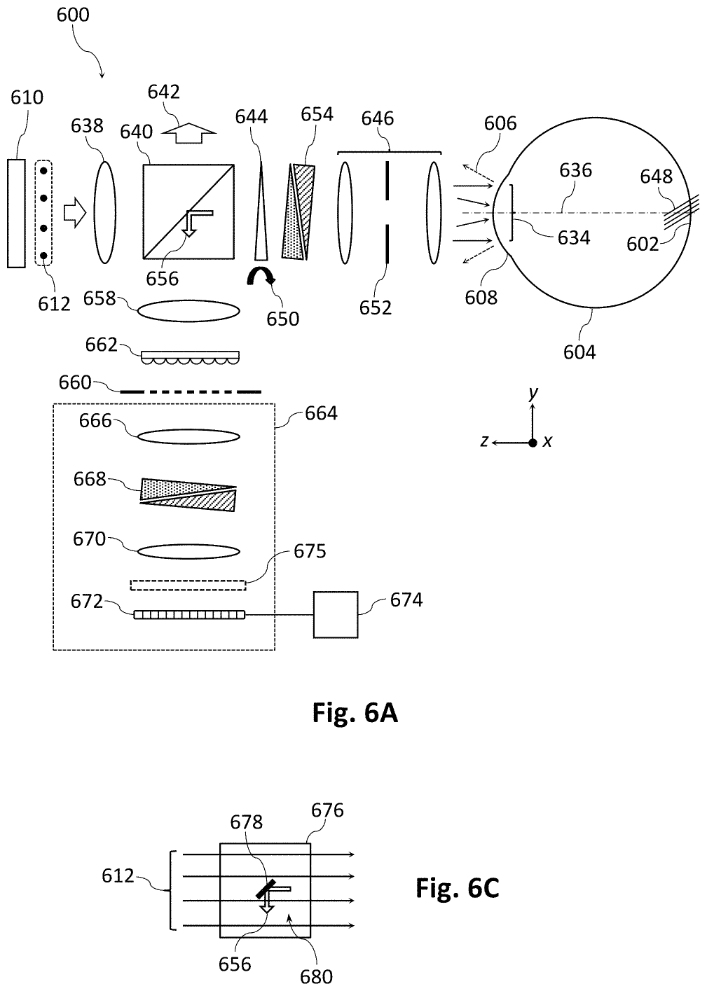

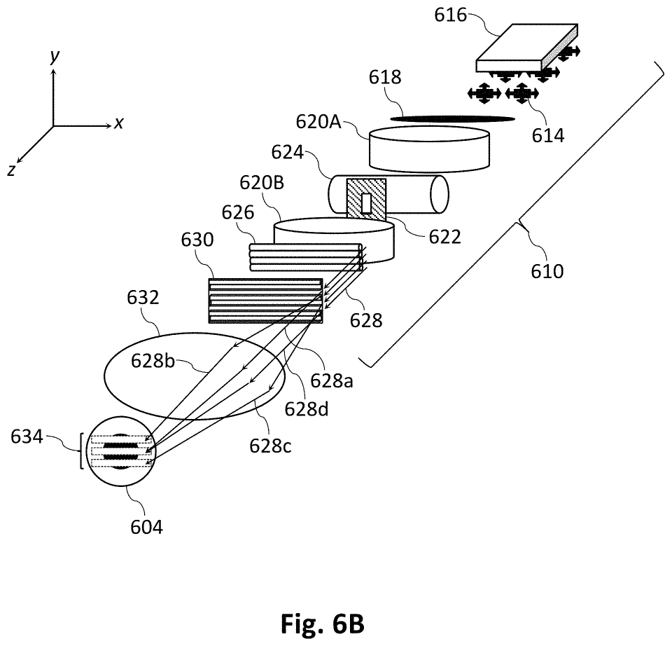

FIG. 6A depicts in schematic plan view an apparatus 600 for in-vivo wide field hyperspectral imaging of the retina 602 of an eye 604, according to another embodiment of the present invention. The apparatus 600 is configured for linear confocal imaging of the retina 602 with rejection of light 606 specularly reflected from the cornea 608. The rejection of specular reflections, together with the ability to illuminate the retina only at required areas, provides improved signal-to-noise ratio compared to conventional hyperpectral imaging systems.

An important component of the apparatus 600 is an illuminator 610 configured to produce an array of thin illumination stripes 612 for projection onto the retina 602. In the plan view of FIG. 6A these illumination stripes 612 extend into the page (x-axis). The bandwidth of the illuminator 610 may be chosen to provide a wavelength range covering selected spectral reflection or absorption features of the retina, or more generally of an object to be analysed. A construction of a suitable illuminator 610 according to certain embodiments is described with reference to the schematic oblique view shown in FIG. 6B. An array of blue LEDs 614 illuminates a thin plate 616 of Ce-YAG or other phosphor of a desired spectral range, to produce a stripe 618 of broadband light that is spatially incoherent to a large extent, at least in the long axis, defined here as the x-axis. The use of a low spatial coherence source is advantageous for safety in in-vivo ocular examination because the emitted light cannot be focused to a small high-intensity spot on the retina. Recycling of the LED power may be provided by a high reflectance coating on selected surfaces of the plate 616. The plate may also be surrounded by an intermediate refractive index material such as glass to reduce the numerical aperture of the emitted beam 618 in the y-axis compared to the case with an air-clad plate, with the structure optionally designed to provide singlemode waveguiding in the y-axis. In this example embodiment we will consider a Ce-YAG plate 616 of approximate dimensions 5×5×0.1 mm emitting a beam 618 of initial approximate dimensions 5 mm in the x-axis and 0.1 mm in the y-axis.

The emitted beam 618 is imaged in the x-axis by cylindrical lenses 620A, 620B each having focal length 10 mm and configured in combination to provide no magnification, although they may be configured to provide magnification if desired. In certain embodiments the beam 618 is spatially filtered by one or more apertures 622 to provide an appropriate numerical aperture and to control regions where the light is projected onto the cornea. The beam 618 may also be filtered spectrally, e.g. with a band-pass filter, to limit the wavelength range. A cylindrical lens 624 of focal length 20 mm is positioned to collimate or magnify the beam 618 in the y-axis, and the collimated or magnified beam directed to an array of one-dimensional focusing elements 626 such as an array of cylindrical lenslets on a 200 μm pitch having focal length 1 mm. In a preferred embodiment the cylindrical lenses 620A, 620B and 624 form a 4F/2F system with the cylindrical lenslet array 626 positioned at or near an image plane in the y-axis and at or near a Fourier plane in the x-axis. The lenslets 626 are oriented to focus in the y-axis, producing a plurality of beamlets 628 elongated in the x-axis. In the illustrated embodiment the beamlets proceed to a structured plate 630 configured with a number of facets for steering individual beamlets in selected directions, with the plate preferably positioned at a focal plane of the cylindrical lenslet array 626 so that the beamlets 628 can be redirected to enter the pupil from different directions without changing their position on the retina. In the illustrated example the structured plate 630 is configured such that the peripheral beamlets 628a, 628d propagate without deviation while the central beamlets 628b, 628c are redirected up and down respectively. One or more optical power elements in the source-to-eye path, described below with reference to FIG. 6A but represented here by a single spherical lens 632, project the beamlets 628 onto a plurality of locations 634 on the eye 604. The focusing action of the eye then maps the light from these locations 634 onto elongated locations on the retina corresponding to the position of the beamlets within the beamlet array 628.

Operation of the hyperspectral imaging apparatus 600 will now be described. Returning to FIG. 6A, the illuminator 610 emits an array of thin illumination stripes 612 that correspond to the elongated beamlets 628 formed by the cylindrical lenslet array 626 as shown in FIG. 6B. Each illumination stripe can be considered as a separate spatially incoherent source, with its own angular trajectory in the x- and y-axes as determined by the structured plate 630. In preferred embodiments this plate is designed to steer each illumination stripe such that specular reflections 606 from the cornea 608 occur at sufficiently large angles to the ocular axis 636, e.g. greater than 12 degrees, for them not to be captured. Capture of corneal reflections is also suppressed by virtue of the fact that the elongated beamlets are focused at the retina, not at the cornea. For non-ocular samples, or if corneal reflections are of minor concern, the structured plate 630 may be omitted. The illumination stripes 612 are collimated with a spherical lens 638 then transmitted through a polarising or non-polarising beam splitting cube 640, with reflected power 642 optionally used to provide a reference beam for coherence-resolved imaging or OCT. The transmitted beamlets proceed to an optional angular deflection element 644, shown here as transmissive although it may be reflective, configured to deflect the beamlets in the y-axis with a θₓ angular deflection 650. The beamlets are relayed to a plurality of locations 634 on the cornea 608 by a focal plane relay 646, with the optical power elements of the eye 604 creating an image 648 on the retina 602. For clarity the on-retina image 648 of the illumination stripes 612 is shown in oblique view as a series of parallel lines. Preferably the apparatus 600 is positioned relative to the eye 604 such that the illuminating light is able to pass through an undilated pupil (not shown), unlike in conventional hyperspectral imaging systems. Although the illumination may be clipped by a constricted pupil in the case of misalignment, this will not affect the location of the image 648 on the retina 602. In general a confocal system using visible light and with good resolution on the eye only requires a pupil size of 1 mm. The position of the image 648 on the retina 602 can be tuned by angular adjustment 650 of the deflection element 644.

In certain embodiments the focal plane relay 646 has focal adjustment capability for optimising formation of the on-retina image 648 and may include an aperture 652 for rejection of specularly reflected light 606. This is in addition to the spatial filtering offered by the confocal nature of the imaging system, described below. The optical train may also include a dispersive element 654 for dispersing light in each illumination line 612 over a larger area of the retina 602 for increased patient comfort, and potentially for facilitating registration of subsequent frames of an acquisition. For these purposes the dispersion direction should be at an angle to the long axis of the illumination lines, more preferably substantially perpendicular to the long axis. In the illustrated embodiment the dispersive element 654 comprises a pair of wedges of materials of different wavelength dispersive properties, with a combination of PMMA and fused silica for example providing a cost-effective dispersive element with low nonlinearity of dispersion. Any dispersion imposed on the beamlets on the outward path will be reversed on the return path, so as not to interfere with the passage of the return light through the confocal linear aperture array 660 described below.

Return light 656 scattered, reflected or fluoresced from the retina 602 within the acceptance angle of the relay optics 646 is reflected by the beam splitter 640 and imaged by a spherical lens 658 onto a linear aperture array 660. A cylindrical lenslet array 662 may be included to convert the numerical aperture of each line, i.e. reduce the spot size in the short axis, for optimal capture of the reflected light through the slits of the aperture array 660. The aperture array is an important component of the hyperspectral imaging apparatus 600, since it creates a linear confocal system that rejects light scattered or reflected from points away from the focal plane, which in this embodiment is positioned at or near the retina 602. The return light passed by the aperture array 660 proceeds to a spectrometer 664 for simultaneous detection, over a range of wavelengths, of the return light. The spectrometer comprises a collimating lens 666, a dispersive element 668 and a focusing lens 670 for focusing and directing the dispersed stripes of scattered/reflected/fluoresced light onto a 2-D focal plane array 672 for read out and processing in a computer 674 equipped with suitable machine-readable code. The dispersive element 668 is oriented to disperse the light stripes in the direction substantially perpendicular to their long dimension, and in the illustrated embodiment comprises a wedged pair. In certain embodiments the dispersive element 668 is birefringent, e.g. by choosing crystalline quartz for one of the wedges and an isotropic material for the other, enabling polarisation diverse detection. In another embodiment polarisation diverse detection is provided by the addition of a walk-off plate 675 in the spectrometer 664.

As shown in FIG. 6C, in an alternative embodiment the beam splitting cube 640 is replaced by a spatially selective directional coupling element in the form of an aperture reflection beam splitter 676 having a reflective area 678 and a transmissive area 680, positioned such that a return signal 656 reflects off the reflective area 678 while allowing the illumination, which could be an array of illumination stripes 612 generated by the illuminator 610 shown in FIG. 6B or some other suitable illuminator, to pass through the transmissive area 680. This embodiment provides greater selectivity of the return signal, rejecting high numerical aperture light arising for example from specular reflections or aberrations in the eye 604.

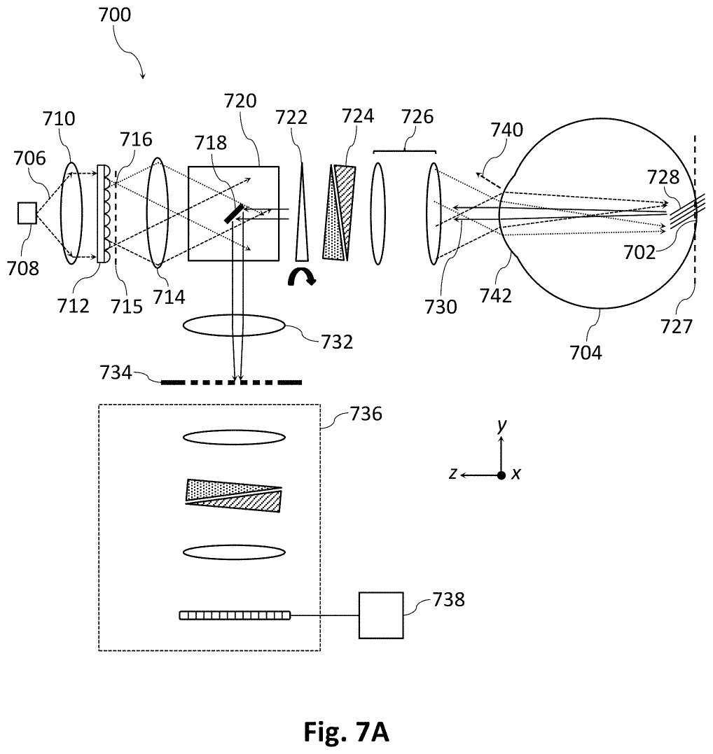

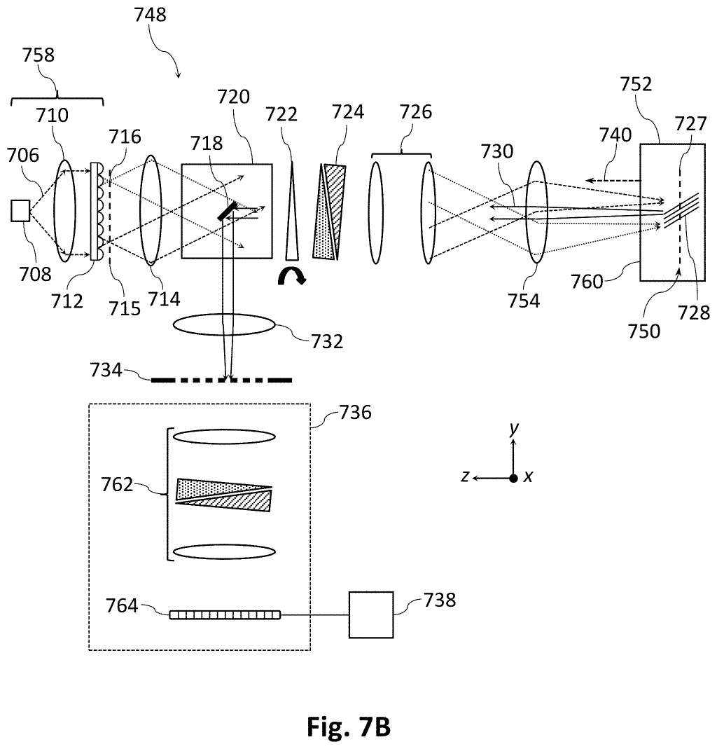

FIG. 7A depicts in schematic plan view an apparatus 700 for in-vivo wide field hyperspectral imaging of the retina 702 of an eye 704, according to another embodiment of the present invention. Light 706 from a broadband optical source 708 such as a SLED is collimated with a spherical lens 710 then passed through a lenslet array 712, which in a preferred embodiment is an array of cylindrical lenslets to produce, at a focal plane 715 of the lenslet array 712, a structured illumination field 716 comprising an array of beamlets elongated in the x-axis. An optical power element in the form of a spherical lens 714 positioned in the far field of the structured illumination field 716 collimates the beamlets, which then proceed towards the eye 704 via an aperture reflection beam splitter 720 similar to that depicted in FIG. 6C, generally with some loss of intensity at the reflective area 718, followed by an optional angular deflection element 722, dispersive element 724 and lens relay 726, before being imaged to a focal plane 727 at or near the retina 702 by the optical power of the eye. In general the image 728 on the retina, shown in oblique view, will comprise a set of possibly overlapping areas corresponding to the wavelength-dispersed beamlets of the structured illumination field 716 if the dispersive element 724 is present, or a set of multi-wavelength lines if the dispersive element is absent.

Light 730 reflected, scattered or fluoresced from the illuminated lines or areas of the image 728 within the acceptance angle of the reflective area 718 of the aperture reflection beam splitter 720 is focused by a lens 732 and spatially filtered with a linear aperture array 734 then analysed spectrally by a spectrometer 736 and a computer 738 as described previously. Reflections 740 from the cornea 742 are substantially excluded from the spectrometer 736 by the fact that the numerical apertures corresponding to the transmissive and reflective portions of the aperture reflection beam splitter 720 converge only at the focal plane 727 positioned at or near the retina 702. This focal plane 727 is conjugate to the focal plane 715 of the lenslet array 712 and the linear aperture array 734. A cylindrical lenslet array may be provided in front of the linear aperture array 734 to improve the collection efficiency by allowing a transformation of the numerical aperture or spot size.

FIG. 7B depicts in schematic plan view an apparatus 748 for hyperspectral confocal imaging of a sub-surface region 750 of an object 752 that may for example be the cornea or retina of an eye or a non-ocular sample. Sub-surface regions that may be of interest include the endothelium of the cornea and the retinal nerve fibre layer. The apparatus 748 is a variation of the apparatus 700 shown in FIG. 7A, modified with the inclusion of an objective lens 754 for projecting the illumination onto a target sub-surface region 750 and for collecting light 730 reflected, scattered or fluoresced from the imaged illumination field 728. The apparatus 748 may be useful for food inspection, e.g. for analysis of layers of fruit a short distance below the surface, while suppressing reflections from the surface.

A broadband optical source 708, lens 710 and cylindrical lenslet array 712 in combination form an illuminator 758 for forming, at a focal plane 715 of the lenslet array 712, a structured illumination field 716 comprising an array of beamlets elongated in the x-axis. An optical power element in the form of a spherical lens 714 positioned in the far field of the structured illumination field 716 collimates the beamlets, which then proceed towards the object 752 via an aperture reflection beam splitter 720 followed by an optional angular deflection element 722, dispersive element 724 and lens relay 726, before being imaged by the objective 754 to a focal plane 727 at the target sub-surface region 750. The axial depth of the imaged illumination field 728 within the object 752 will depend, for a given distance between the apparatus 748 and the object, on the details of the optics in the illumination train. As in the previous embodiment shown in FIG. 7A the imaged illumination field 728, shown here in oblique view. may comprise a set of possibly overlapping areas or a set of multi-wavelength lines, depending on whether a dispersive element 724 is present.

The spatially selective directional coupling element 720 with its reflective portion 718 is positioned so as to direct at least a portion of the structured illumination field 716 towards the object 752 and to direct at least a portion of the reflected, scattered or fluoresced light 730 towards a spatially structured confocal aperture in the form of a linear aperture array 734.

Light passed by the linear aperture array proceeds to a spectrometer 736 comprising a dispersive optical relay 762 that projects a wavelength-dispersed image of the linear aperture array onto a 2-D sensor array 764 for read out and processing in a computer 738 equipped with suitable machine-readable code. Similar to the embodiment shown in FIG. 7A, reflections 740 from surfaces in front of or behind the target sub-surface region 750, in particular from the front surface 760 of the object 752, are suppressed by the fact that the numerical apertures corresponding to the transmissive and reflective portions of the aperture reflection beam splitter 720 converge only at the focal plane 727 positioned at the target sub-surface region 750. As in FIG. 7A the focal plane 727 is conjugate to the focal plane 715 of the lenslet array 712 and the linear aperture array 734, and a cylindrical lenslet array may be provided in front of the linear aperture array 734 to improve the collection efficiency.

In alternative embodiments the apparatus 700, 748 depicted in FIGS. 7A and 7B include a 2-D array of spherical lenslets instead of the cylindrical lenslet array 712, in which case the structured illumination field 716 will comprise a 2-D array of beamlets. In these embodiments the imaging side of the apparatus includes a 2-D aperture array in place of the linear aperture array 734, with the orientation of the 2-D aperture array with respect to the dispersion axis of the dispersive optical relay 762 preferably chosen to ensure that each dispersed beamlet is mapped onto a unique set of pixels of the 2-D sensor array 764.

The various apparatus described above have been depicted in reflective configurations, e.g. for in-vivo retinal imaging. However it will be appreciated that in general the inventive principles could also be applied to transmissive configurations where light scattered or fluoresced from a partially transmissive sample is captured.

Although the invention has been described with reference to specific examples, it will be appreciated by those skilled in the art that the invention may be embodied in many other forms.

1. An apparatus for imaging an eye, said apparatus comprising:
an illumination system comprising an optical source for illuminating, with a multi-wavelength optical beam, a volume of the eye, said volume to be imaged in three spatial dimensions, said volume having an elongated lateral cross-sectional area with a short axis, a long axis and an aspect ratio defined by the ratio of the long axis to the short axis;
an optical system for capturing and anisotropically transforming light scattered or reflected from the illuminated volume;
one or more beam splitters for splitting light emitted from said optical source into a reference beam and said multi-wavelength optical beam, and for combining said reference beam with the captured light;
a spatial sampling element for sampling the anisotropically transformed captured light in a first dimension; and
a measurement system comprising a two-dimensional sensor array for simultaneous capture of phase and amplitude information over a range of wavelengths of the captured light sampled by said spatial sampling element.

2. The apparatus according to claim 1, wherein said optical system is configured to cooperate with the optical power elements of the eye, for imaging the retina of the eye.

3. The apparatus according to claim 2, wherein said measurement system is configured to capture phase and amplitude information in first and second frames of said two-dimensional sensor array, for measurement of changes due to blood flow in the illuminated volume of the retina.

4. An optical imaging apparatus comprising:
an illumination system comprising an optical source for illuminating, with a multi-wavelength optical beam, a volume of an object, said volume to be imaged in three spatial dimensions, said volume having an elongated lateral cross-sectional area with a short axis, a long axis and an aspect ratio defined by the ratio of the long axis to the short axis;
an optical system for capturing and anisotropically transforming light scattered or reflected from the illuminated volume;
one or more beam splitters for splitting light emitted from said optical source into a reference beam and said multi-wavelength optical beam, and for combining said reference beam with the captured light;
a spatial sampling element for sampling the anisotropically transformed captured light in a first dimension; and
a measurement system comprising a two-dimensional sensor array for simultaneous capture of phase and amplitude information over a range of wavelengths of the captured light sampled by said spatial sampling element.

5. The apparatus according to claim 4, wherein said optical system is configured such that, in use, the aspect ratio of the anisotropically transformed light at said spatial sampling element is less than the aspect ratio of the lateral cross-sectional area of said illuminated volume.

6. The apparatus according to claim 4, wherein said spatial sampling element is positioned for Fourier plane sampling of said captured light.

7. The apparatus according to claim 4, wherein said spatial sampling element comprises a cylindrical lenslet array or a linear aperture array.

8. The apparatus according to claim 4, wherein said optical system and said spatial sampling element are configured such that, in use, the captured light sampled by said spatial sampling element is projected onto a substantial portion of said two-dimensional sensor array, said substantial portion comprising at least 100 pixels in each dimension.

9. The apparatus according to claim 4, wherein said optical source is at least partially spatially incoherent.

10. The apparatus according to claim 9, comprising an aperture for adjusting the spatial coherence of the light emitted from said optical source.

11. The apparatus according to claim 9, wherein said optical source or said aperture are selected such that said multi-wavelength optical beam is substantially spatially coherent across said short axis of said lateral cross-sectional area.

12. (canceled)

13. The apparatus according to claim 4, wherein said measurement system comprises a dispersive element for dispersing the captured light in the direction substantially parallel to the sampling dimension of said spatial sampling element.

14. The apparatus according to claim 4, wherein said optical system comprises a series of cylindrical lenses forming a 4F relay system in the direction of said long axis and a 2F relay system in the direction of said short axis.

15. The apparatus according to claim 4, wherein said illumination system is configured such that, in use, the aspect ratio of said elongated lateral cross-sectional area is at least ten.

16. (canceled)

17. The apparatus according to claim 15, wherein said illumination system is configured such that, in use, the aspect ratio of said elongated lateral cross-sectional area is at least fifty.

18. (canceled)

19. The apparatus according to claim 4, comprising one or more optical power elements for re-sizing said reference beam so as to increase the overlap between said reference beam and the captured light.

20. The apparatus according to claim 4, comprising a computer for processing the phase and amplitude information to construct a three-dimensional image of an optical characteristic of said object over said illuminated volume.

21. The apparatus according to claim 20, wherein said optical characteristic is selected from the group comprising phase, reflectivity, refractive index, refractive index changes and attenuation.

22. The apparatus according to claim 20, wherein said measurement system comprises a polarisation separation element for capturing phase and amplitude information for at least first and second polarisation states of the captured light.

23. The apparatus according to claim 22, wherein said optical characteristic comprises birefringence or degree of polarisation.

24. The apparatus according to claim 20, wherein said computer is configured to apply a focusing or aberration correction function to the phase and amplitude information.

25. The apparatus according to claim 4, wherein said apparatus is configured to move the illuminated volume in a direction substantially perpendicular to the long axis of its elongated lateral cross-sectional area, for imaging larger volumes of said object.

26.-47. (canceled)

48. A method for optical imaging, said method comprising the steps of:
illuminating, with a multi-wavelength optical beam, a volume of an object, said volume to be imaged in three spatial dimensions, said volume having an elongated lateral cross-sectional area with a short axis, a long axis and an aspect ratio defined by the ratio of the long axis to the short axis;
capturing and anisotropically transforming light scattered or reflected from the illuminated volume;
splitting light emitted from an optical source into a reference beam and said multi-wavelength optical beam;
combining said reference beam with the captured light;
sampling the anisotropically transformed captured light in a first dimension; and
simultaneously capturing, with a measurement system comprising a two-dimensional sensor array, phase and amplitude information over a range of wavelengths of the sampled anisotropically transformed captured light.

49.-51. (canceled)

52. The apparatus according to claim 4, wherein said apparatus is configured such that, in use, said captured light is sampled in said first dimension by said spatial sampling element at a Fourier plane and sampled in a second dimension
